(19) United States
(12) Patent Application Publication
(10) Pub. No.: US 2016/0175444 A1
(43) Pub. Date: Jun. 23, 2016

(54) Treatment for non-genetic biological disease

(71) Applicant: Michael Lewis Moravitz, Fairfax, VA (US)

(72) Inventor: Michael Lewis Moravitz, Fairfax, VA (US)

(21) Appl. No.: 14/998,793

(22) Filed: Feb. 18, 2016

(51) Int. Cl. A61K 45/00

(52) U.S. Cl. CPC A61K 45/00

(57) ABSTRACT

New Treatments for Illnesses Thought to be Genetically-based.

Treatment options can be developed based on the idea that sexual relations exchanges fluids other than semen and conditions in the mother and any offspring may be related. It is known, of course, that babies can be addicted to substances in their mothers.

However, men may affect the health of their children by any diseases that may enter the female's vagina and then affect the development of the child. This may mean much less emphasis on the search for genetic reasons for certain diseases, including certain conditions such as scrofula, roseola, and acne. There may be a number of conditions—thought to be genetic—that develop in children based on diseases, poisons, or chemicals transferred through sexual intercourse and also the mother alone. This does not mean there is no hope for the parents or child: rather an opportunity for improving the health of children overall. Research on this idea is needed to develop new treatment options.

1) Builds on Patents for Antibiotics;

2) and Patents for Other Medicines for Supposed Genetically-based Illnesses.
